(19) United States
(12) Patent Application Publication
(10) Pub. No.: US 2024/0412822 A1
(43) Pub. Date: Dec. 12, 2024

(54) METHOD FOR INTERPRETING INTER-TUMOR AND INTRA-TUMOR HETEROGENEITY IN SMALL CELL LUNG CANCER

(71) Applicant: Cancer Hospital Chinese Academy of Medical Sciences, Beijing (CN)

(72) Inventors: Lin YANG, Beijing (CN); Zicheng ZHANG, Wenzhou, Zhejiang (CN); Meng ZHOU, Wenzhou (CN)

(21) Appl. No.: 18/591,857

(22) Filed: Feb. 29, 2024

(30) Foreign Application Priority Data
Jun. 6, 2023 (CN) 202310657772.1

(51) Int. Cl. G16B 40/30; G16H 50/20

(52) U.S. Cl. CPC G16B 40/30; G16H 50/20

(57) ABSTRACT

The disclosure discloses a method for an interpreting inter-tumor and intra-tumor heterogeneity in a small cell lung cancer (SCLC), including following steps: step 1, calculating heterogeneity characteristics of ROI samples based on a digital space transcriptomics technology; step 2, mapping the ROI samples to a patient level and providing a heterogeneity typing mode according to a prognosis stratification for typing; step 3, analyzing a heterogeneity mechanism and finding core gene sets determining a heterogeneity typing; step 4, constructing a tumor heterogeneity index model THIM based on the core gene sets and carrying out a THIM scoring processing; and step 5, a mapping application and a verification of THIM intelligent model.

### CROSS-REFERENCE TO RELATED APPLICATIONS

This application claims priority to Chinese Patent Application No. 202310657772.1, filed on Jun. 6, 2023, the contents of which are hereby incorporated by reference.

### TECHNICAL FIELD

The disclosure relates to a technical field of biology, and in particular to a method for interpreting an inter-tumor and intra-tumor heterogeneity in a small cell lung cancer (SCLC).

### BACKGROUND

Small cell lung cancer (SCLC) is a highly heterogeneous cancer with aggressive progression and poor prognosis. At present, subtypes based on neuroendocrine (NE) differentiation and transcription factor (TF) have been proposed, but the prognostic significance and the clinical treatment relevance are still controversial. Recent studies have shown that intra-tumor and inter-tumor heterogeneity (ITH) is related to the biological behavior and the treatment vulnerability of multiple malignant tumors. However, the definition and interpretation of the clinical relevance of ITH in SCLC are still unclear.

The clinical treatment strategy of SCLC has made slow progress for decades. Although the recent chemotherapy combined with immunotherapy has shown promising benefits, the clinical beneficiaries are limited and there is no effective predictor of curative effect. NE typing and TF typing modified that the conventional understanding that SCLC is a homogeneous tumor characterized by a co-mutation of TP53 and RB1. At present, SCLC is considered with a characterization of complex heterogeneity, which not only shows that patients with the same clinical stage have different prognosis, but also shows subtype transformation during tumor progress or treatment.

However, the current understanding of SCLC ITH is mainly derived from the transcriptome sequencing or single cell quasi-time series analysis of cell lines/mouse models or a small number of human tumor fresh samples, and there is a lack of pathological heterogeneity observation based on clinicopathological formalin-fixed and paraffin-embedded (FFPE) samples, and there is also a lack of research on quantitative indicators related to ITH. The definition of tumor heterogeneity and its clinical relevance are still unclear; therefore, the present disclosure provides a method for interpreting inter-tumor and intra-tumor heterogeneity in small cell lung cancer to solve the problems existing in the prior art.

### SUMMARY

In view of the above problems, the present disclosure provides a method for an interpreting inter-tumor and intra-tumor heterogeneity in a small cell lung cancer (SCLC), the method for interpreting the inter-tumor and intra-tumor heterogeneity in the SCLC deciphers an intra-tumor and inter-tumor heterogeneity (ITH) of SCLC on pathological sections, may accurately compare gene expression, biological process and immune infiltration in different regions of the same tumor without microdissection, and proves that the prognosis of highly heterogeneous subgroups is poor. At the same time, a tumor heterogeneity index model (THIM) intelligent model is established, which may predict the prognosis and immunotherapy response of the SCLC, improve the clinical risk stratification and molecular classification, and contribute to the stratified management of patients in limited stage after operation and the evaluation of immunotherapy efficacy in patients in advanced stage.

In order to achieve the purpose of the disclosure, the disclosure is realized by a following technical scheme: a method for interpreting the inter-tumor and intra-tumor heterogeneity in the SCLC includes following steps:
step 1, selecting a plurality of Regions of Interest (ROI) on a same case section based on digital space transcriptomics, and then extracting Heterogeneity characteristics of the ROI samples according to four calculation methods of a spatial physical distance of the ROI, and dividing the heterogeneity characteristics of the ROI samples into a high heterogeneity, a medium heterogeneity and a low heterogeneity;
step 2, mapping the heterogeneity characteristics of the ROI samples back to a patient level, defining tumor heterogeneities among patients, and dividing the tumor heterogeneities into a high complex (HC) group and a middle-low complex (ML) group, then carrying out a survival analysis and a score analysis on the HC group and the ML group, and comparing with a conventional transcription factor (TF) typing and a conventional neuroendocrine (NE) typing;
step 3, comparing specifically up-regulated mRNA in the HC group and the ML group by transcriptomics functional differential expression analysis, and carrying out a biological function annotation, then analyzing a difference of immune microenvironment characteristics between the HC group and the ML group by using an immune infiltration evaluation algorithm, and finding 10 core gene sets determining a tumor heterogeneity typing by a machine learning algorithm;
step 4, selecting and constructing a THIM based on the 10 core gene sets in the step 3, and then dividing the heterogeneity characteristics of the ROI samples into a training set, a first test set and a second test set according to a ratio of 75%:15%:15% to carry out a scoring process on the THIM; and
step 5, grouping ROI samples according to the scoring process in the step 4, and mapping groups to the patient level for prognosis analysis to obtain a prognosis result, thus completing a verification of the THIM intelligent model.

According to a further improvement scheme: in the step 1, after obtaining the heterogeneity characteristics of the ROI samples, calculating CV-score of each gene in a whole gene range according to a variability scoring formula, and sorting according to a criterion from high to low; selecting top 200 highly mutated genes as candidate features, carrying out an unsupervised hierarchically clustering on the heterogeneity characteristics of the ROI samples, then naming according to distribution trends of ITH-score, C-score and CV-score respectively to obtain three ROI groups with different heterogeneities: a high heterogeneity group H-H, a middle heterogeneity group M-H and a low heterogeneity group L-H, and there is no significant difference in an actual spatial physical distance between the three groups, and there is no correlation between the actual spatial physical distance and the C-score, so as to prove a heterogeneity of ROI independent of noise generated by manual point selection.

According to a further improvement scheme: the survival analysis of the HC group and the ML group in the step 2 shows that there is no significant difference between clinical feature combinations of two groups of patients, and heterogeneous groups are identified as independent clinicopathological features through a combined prognosis analysis with clinicopathological features, that is, the prognosis of the HC group is worse than the prognosis of the ML group.

According to a further improvement scheme: in the step 2, the score analysis of the HC group and ML group is carried out by using the ITH-score, the C-score and the CV-score at a transcription level, and results with significant heterogeneity differences between patients of the HC group and the ML group are obtained.

According to a further improvement scheme: in the step 2, grouping modes of the HC group and the ML group are compared with conventional TF typing and NE typing, and it is concluded that the grouping modes of the HC group and the ML group are superior to the conventional TF typing and NE typing in distinguishing prognosis of patients.

According to a further improvement scheme: in the step 3, after analyzing a difference of immune microenvironment characteristics between the HC group and the ML group by using an immune infiltration evaluation algorithm, a result is obtained: an infiltration degree of all T cells and CD8+ T cells in ROI samples in the ML group is significantly higher than that in HC group.

According to a further improvement scheme: in the step 4, before selecting and constructing the THIM model, a feature selection is carried out on 129 differentially expressed genes (DEGs) by using an automatic encoder and is repeated for 500 times, and finally the top 10 genes are selected as candidates, and 10 groups of candidate genes are verified by immunofluorescence staining, and finally the model is selected and constructed according to the 10 groups of candidate genes after verification, where the 10 groups of candidate genes include 4 HC group-specific genes and 6 ML group-specific genes.

According to a further improvement scheme: in the step 5, when scoring, a group with a THIM score greater than 0.45 is defined as the high heterogeneity group, and a group with a THIM score of not more than 0.45 is defined as the low heterogeneity group; and in the step 5, the prognosis analysis result is as follows: the prognosis of the high heterogeneity group is worse than the prognosis of the low heterogeneity group.

The disclosure has following beneficial effects: The intra-tumor heterogeneity of SCLC is deciphered on pathological sections, successfully mapped to the patient level, and the inter-tumor heterogeneity index grouping model is defined. The gene expression, biological process and immune infiltration in different regions of the same tumor are accurately compared without microdissection, and the prognosis of highly heterogeneous subgroups is confirmed to be poor.

At the same time, after using 10 core differentially expressed genes for ITIH scoring, the invention establishes a THIM intelligent model based on the heterogeneity among different tumors at the patient level, and realizes the prediction of the prognosis and immunotherapy response of SCLC, which may not only significantly separate the prognosis of patients, but also better predict the immunotherapy efficacy than PD-1/PDL1 proved on external independent data sets, improve the risk stratification and molecular classification related to clinic, and contribute to the postoperative stratified management of patients in limited period and the efficacy evaluation of combined immunotherapy for patients in late period.

### BRIEF DESCRIPTION OF THE DRAWINGS

FIG. 1 is a flow chart of a method of the present disclosure.

FIG. 2 is a schematic diagram of feature selection of 129 DEGs by using an automatic encoder.

FIG. 3 is a schematic flow chart from the screening process of the core gene of the present disclosure to the construction of a tumor heterogeneity index model (THIM) intelligent model.

FIG. 4 is a Sankey diagram of the comparation across the ITH (intra-tumor and inter-tumor heterogeneity) subgroups, the transcription factor (TF) subgroups and the neuroendocrine (NE) subgroups.

### DETAILED DESCRIPTION OF THE EMBODIMENTS

In order to deepen the understanding of the disclosure, the disclosure is further described in detail with an embodiment, the embodiment is only used to explain the disclosure and does not constitute a limitation on the protection scope of the disclosure.

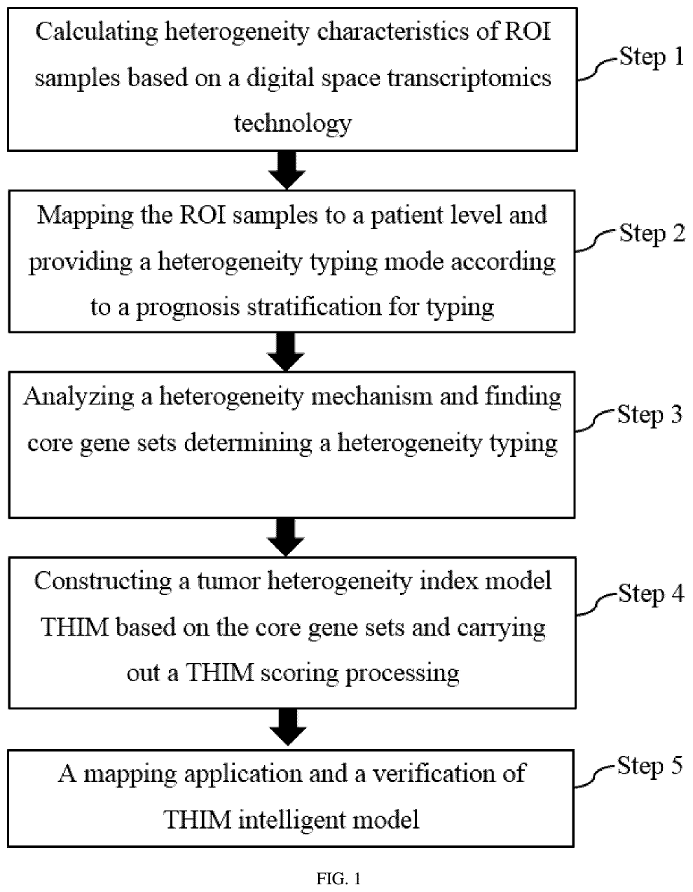

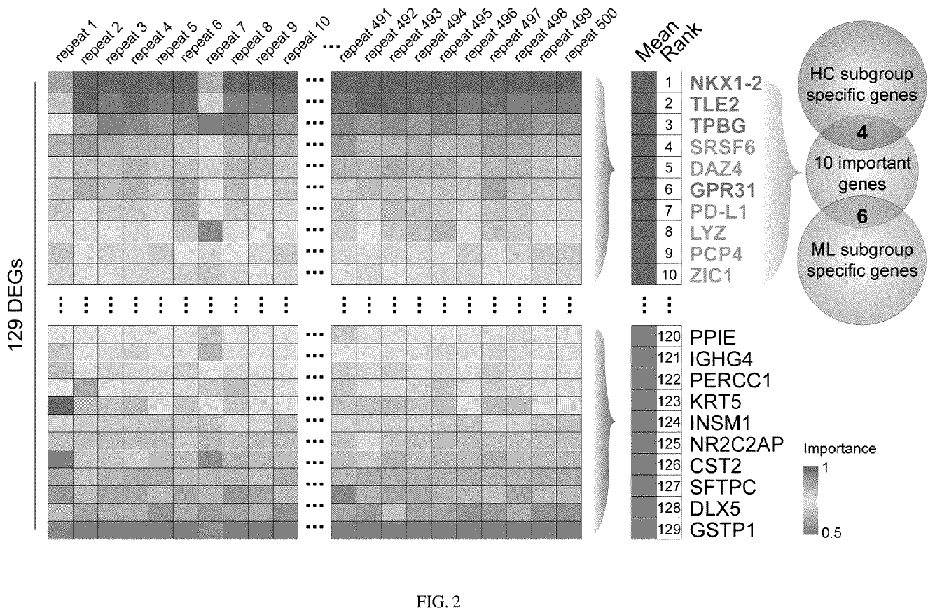

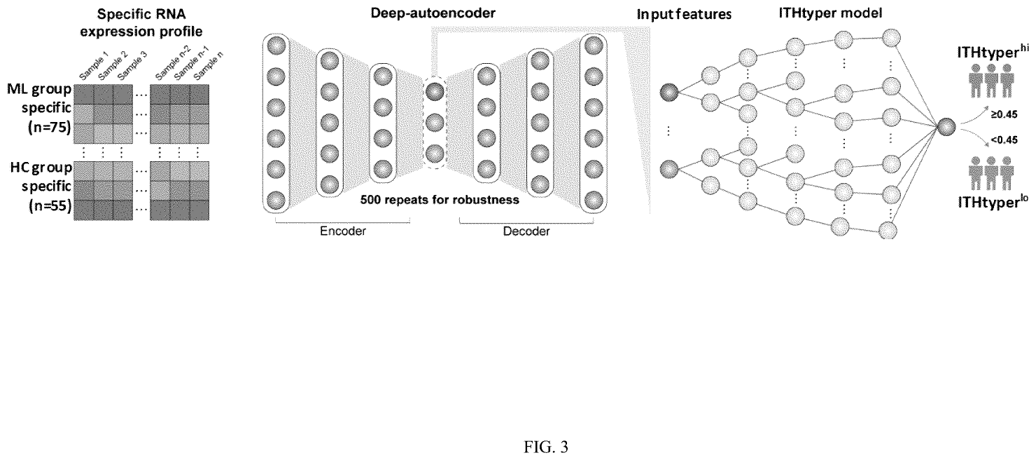

According to FIG. 1, FIG. 2 and FIG. 3, this embodiment provides a method for interpreting an inter-tumor and intra-tumor heterogeneity in a small cell lung cancer (SCLC), including following steps:
Step 1, digital space transcriptome analysis and position information reveal an objective existence of intra-tumor and inter-tumor heterogeneity (ITH) in SCLC;
based on transcriptomes of digital space transcriptomics and four calculation methods of a spatial physical distance, a correlation between Region of Interest (ROI) and ROI and their respective heterogeneous scores are revealed from a transcriptome level, the real distance between ROI points in the same patient is calculated from the spatial physical level, a real contour of tumor tissue, a real distribution of ROI points and dyed pictures of whole transcriptome atlas (WTA) data are obtained, and the heterogeneity characteristic groups of ROI samples are obtained.

The true distribution of the physical distance of ROI in the candidate samples shows that the distribution of ROI points is diverse, including both nearby points and far-end areas;
after the ROI samples are obtained, CV-score of each gene in the whole gene range is calculated according to a variability scoring formula, and sorted according to a criterion from high to low. The top 200 highly mutated genes are selected as candidate features, and an unsupervised hierarchically clustering is carried out on the ROI samples, and then named according to distribution trends of ITH-score, C-score and CV-score respectively. Three groups of ROI subgroups with different heterogeneity are obtained: H-H high heterogeneity group, M-H medium heterogeneity group and L-H low heterogeneity group. There is no significant difference in the actual spatial physical distance (SPD) among the three subgroups, and there is no correlation between SPD and C-score, so as to prove a heterogeneity of ROI independent of the noise generated by manual point selection.

Step 2, ROI typing components reveal the heterogeneity between tumors related to prognosis and treatment results.

The ROI samples are mapped back to the patient level, and the patients are divided into a high complex (HC) subgroup and a middle-low complex (ML) subgroup according to the prognosis of patients, and then the heterogeneity between the HC subgroup and the ML subgroup is verified according to the ITH-score, C-score and CV-score at the transcriptome level.

Then, the heterogeneity between the HC subgroup and the ML subgroup is verified according to the transcriptome level indicators ITH-score, C-score and CV-score, and it is determined that there is no significant difference between clinical feature combinations of the two groups, and heterogeneous groups are identified as independent clinicopathological features through a combined prognosis analysis with clinicopathological features.

The prognostic analysis shows that the prognosis of patients in the ML subgroup (whether Overall Survival (OS) or Disease Free Survival (DFS), including 3-year and 5-year survival rate) is significantly better than the prognosis of patients in the HC subgroup.

Further combining the information of survival status and recurrence status, it is revealed that there are no death and recurrence events in the ML subgroup, while the proportion of death events and recurrence events in the HC subgroup is 50% and 61.1%.

Step 3, an interaction between the ITH subtype and conventional subtypes of SCLC.

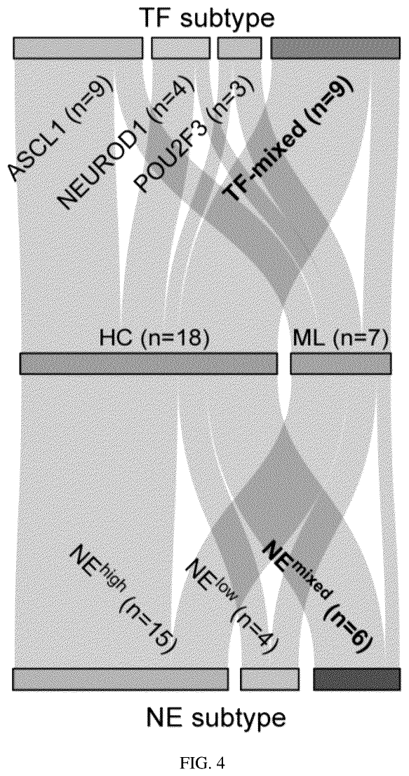

The advantages of heterogeneous typing over the conventional TF typing and the NE typing are compared and verified, that is: the heterogeneous typing may better distinguish the prognosis and stratify the prognosis of patients;
specifically, according to the traditional subtyping based on the expression of four transcriptors (ASCL1, NEUROD1, POU2F3, YAP1, ANPY in short), the ROI samples are clustered and divided into three subtypes, A, P and N, and the ROI regions of different (TF) transcription factor subgroups are mapped to the patient level, so that the patients with simple subtypes A, N and P and patients with mixed TF complex are obtained. The adaptability and advantages of the heterogeneous typing proposed in the disclosure and the conventional TF typing are compared, showing that the conventional TF typing suitable for cell lines also has complex heterogeneity in tumor tissues of real patients;
a cluster division is carried out on the ROI samples according to the classification criteria of NE (Neuroendocrine differentiation) score subtypes, and the ROI samples with NE score greater than 0 are defined as subgroups with high NE, and the ROI samples with NE score less than 0 are defined as subgroups with low NE; then, ROI sample areas of different NE subgroups are mapped to the patient level, and the results are similar to TF subgroups, patients with simple high NE and low NE are obtained, and patients with NE complex are also obtained. This result also shows that the NE typing is also highly heterogeneous in large tissues of patients, confirming the adaptability of the heterogeneous classification of the disclosure to the conventional NE classification and the advantages of the heterogeneous classification proposed of the disclosure;
when analyzing the mapping results, the correlation between ITH (intra-tumor and inter-tumor heterogeneity) subgroups, the TF subgroups and the NE subgroups is compared and analyzed by Sankey Diagram (FIG. 4), and it is found that both TF subgroups and NE subgroups are distributed in the HC subgroup and the ML subgroup in a balanced way, with no statistical difference, indicating that there are patients with simple A, N and P subtypes and patients with high and low NE in HC subgroup, and there are also patients with TF complex and NE complex, therefore, it is proved that the heterogeneity typing proposed in the disclosure only adapts to the conventional typings, but also explain the conventional typings.

Step 4, the transcriptome functional analysis reveals the changes of CD8+T cell infiltration of the ITH subtype.
mRNA specifically up-regulated in the HC subtype and the ML subtype is obtained by the differential expression analysis of transcriptomics functions, and the biological function is annotated.

ML subgroup samples are all significantly enriched in immune-related functions: IFN-γ response, regulation of α-βT cell differentiation, innate immune response in mucosa, negative regulation of mRNA metabolism, production of interleukin-8, etc.

HC subgroup samples are significantly enriched to: positive regulation of phospholipase C-activated G protein-coupled receptor signaling pathway, DNA replication-dependent chromatin assembly, regulation of neural precursor cell proliferation, positive regulation of neural precursor cell proliferation, and gastrointestinal morphogenesis.

ANXA1, with anti-inflammatory activity, plays a role in the down-regulation of glucocorticoid-mediated inflammatory response in the early stage (through similarity); ANXA1 promotes adaptive immune response and regulates the differentiation and proliferation of activated T cells by enhancing the signal cascade triggered by T cell activation, promotes T cells to differentiate into Th1 cells and negatively regulates T cells to differentiate into Th2 cells, but has no effect on unstimulated T cells, promotes inflammation regression and wound healing, and enhances the release of CXCL2 through the action of neutrophil N-formyl peptide receptor.;

An enrichment evaluation is further carried out by using tumor-promoting immunity, anti-tumor immunity, angiogenesis/fibroblasts and EMT/tumor proliferation gene sets. It is found that anti-tumor cytokines are significantly enriched in ML subtype, while the characteristics of B cells, NK cells and neutrophils are significantly enriched in the HC subtype.

Then the immune microenvironment composition in the ROI region of CD8+T between the two subtypes is analyzed by using immune infiltration evaluation algorithms of CIBERSORT, MCPCOUNTER and TIMER, and it is found that the infiltration degree of all T cells and CD8+T cells in the ROI samples in the ML subgroups is significantly higher than that in the HC subgroups, the heterogeneity mechanism is explained from the perspective of immune microenvironment, and a core gene set determining the heterogeneity typing is found.

According to the above, the correlation analysis of ANXA1 RNA expression and protein expression related to T cells and CD8+T cells shows that there is a significant correlation between the ANXA1 RNA expression and protein expression related to T cells and CD8+T cells in the ML subgroup, but there is no significant correlation between the ANXA1 RNA expression and protein expression related to T cells and CD8+ T cells in the HC subgroup.

Step 5, the tumor heterogeneity index model (THIM) reveals the prognosis and treatment response.

THIM is constructed based on the core gene set, and then the heterogeneity characteristics of the ROI samples are divided into a training set and a test set according to a division ratio of 70%:30%. The training set and the test set are used to train and test the THIM, and an THIM intelligent model is obtained. The classification performance of six methods and models is compared, among which XGboost (extreme gradient lifting decision tree) wins, verifying a clinical value of the THIM in clinical patient grouping.

Before selecting and constructing the THIM, a feature selection is carried out on 129 differentially expressed genes (DEGs) by using an automatic encoder and is repeated for 500 times to ensure the robustness. Finally, the top 10 genes are selected as candidates, as shown in FIG. 2, and 10 groups of candidate genes are verified by immunofluorescence staining. Finally, the model is selected and constructed according to the 10 groups of candidate genes after verification, the screening process of core genes to the construction of the THIM intelligent model is shown in FIG. 3, where the 10 groups of candidate genes include 4 HC group-specific genes and 6 ML group-specific genes.

Step 6, THIM intelligent model is applied to the training set and the test set, and then divided into groups after THIM scoring, and the groups are mapped to the patient level for prognosis analysis to obtain prognosis results, thus completing the verification of THIM intelligent model;

When scoring for grouping, a group with a THIM score greater than 0.45 is defined as THIM high ROI (high heterogeneity), and a group with a THIM score of not more than 0.45 is defined as THIM low ROI (low heterogeneity). The THIM grouping of ROI is mapped to the patient level for prognosis analysis, and the results show that the prognosis (survival curve of OS/DFS and occurrence of OS/DFS events) of patients with high THIM group is significantly worse than the prognosis of patients with low THIM group.

An external independent test set George & Jiang cohort is used to verify the prognosis.

THIM score/label may effectively predict the immunotherapy response, and its performance is better than that based on the expression level of PD-1 and PDL1 (Roper cohort: the data excludes an atypical sample), and the THIM score in ICB treatment response group is obviously lower. Finally, it is found that all patients in THIM high group have no response to anti-PDL1 immunotherapy.

The basic principle, main features and advantages of the present disclosure have been shown and described above. It should be understood by those skilled in the art that the present disclosure is not limited by the above-mentioned embodiments, and what is described in the above-mentioned embodiments and descriptions only illustrates the principles of the present disclosure. Without departing from the spirit and scope of the present disclosure, there will be various changes and improvements in the present disclosure, which fall within the scope of the claimed disclosure. The scope of the present disclosure is defined by the appended claim and their equivalents.

1. A method for interpreting an inter-tumor and intra-tumor heterogeneity in a small cell lung cancer, comprising following steps:
step 1, selecting ROI samples on a same case section based on digital space transcriptomics, and then extracting heterogeneity characteristics of the ROI samples according to a spatial physical distance between selected points of the ROI samples;
after obtaining the heterogeneity characteristics of the ROI samples, calculating CV-score of each gene in a whole gene range according to a coefficient of variation calculation formula, and sorting according to a criterion from high to low, selecting top 200 highly mutated genes as candidate features, and carrying out an unsupervised hierarchically clustering on the ROI samples based on the candidate features; then grouping and naming the ROI samples according to distribution trends of ITH-score, so as to obtain three ROI groups with different heterogeneities: a high heterogeneity group H-H, a middle heterogeneity group M-H and a low heterogeneity group L-H;
step 2, mapping the ROI groups back to a patient level, defining an inter-tumor heterogeneity and an intra-tumor heterogeneity of patients, and dividing the patients into a HC subgroup and a ML subgroup, then carrying out a survival analysis and a score analysis on the HC subgroup and the ML subgroup, and comparing and verifying the HC subgroup and ML subgroup with a conventional TF typing and a NE typing respectively;
step 3, comparing specifically up-regulated mRNA in the HC subgroup and the ML subgroup by a transcriptome differential expression analysis method, and carrying out a biological function annotation, then analyzing a difference of immune microenvironment characteristics between the HC subgroup and the ML subgroup by using an immune infiltration evaluation algorithm, and finding 10 core gene sets determining a tumor heterogeneity typing by a machine learning algorithm;
step 4, selecting and constructing a tumor heterogeneity index model THIM based on the 10 core gene sets in the step 3, and then dividing the ROI samples into a training set and a test set according to the heterogeneity characteristics of the ROI samples in a ratio of 70%:30%, and carrying out a scoring process on the training set and the test set through the tumor heterogeneity index model THIM; and
step 5, grouping the training set and the test set after scoring in the step 4, and respectively mapping grouping results to the patient level for prognosis analysis, so as to obtain a prognosis result, completing a verification of the tumor heterogeneity index model THIM, and using the tumor heterogeneity index model THIM after verification to interpret the inter-tumor and intra-tumor heterogeneity of the small cell lung cancer; wherein the grouping the training set and the test set after scoring in the step 4, comprises: determining ROI samples in the training set and the test set with a score of the tumor heterogeneity index model THIM greater than 0.45 as a high heterogeneity group, otherwise, as a low heterogeneity group, and in the step 5, the prognosis result is: prognosis of the high heterogeneity group is worse than that of the low heterogeneity group; and
wherein the method further comprises: applying the tumor heterogeneity index model THIM to determine efficacy of combined immunotherapy of patients in late period, thereby performing the combined immunotherapy for the patients in late period.

2. The method for interpreting the inter-tumor and intra-tumor heterogeneity in the small cell lung cancer according to claim 1, wherein in the step 2, after the survival analysis of the HC subgroup and the ML subgroup, no significant difference is shown between clinical feature combinations of two groups of patients, and heterogeneous groups are identified as independent clinicopathological features through a combined prognosis analysis with clinicopathological features.

3. The method for interpreting the inter-tumor and intra-tumor heterogeneity in the small cell lung cancer according to claim 1, wherein in the step 2, the score analysis of the HC subgroup and the ML subgroup is carried out by using the ITH-score and C-score at a transcription level respectively, and results with significant heterogeneity differences between patients of the HC subgroup and patients of the ML subgroup are obtained.

4. The method for interpreting the inter-tumor and intra-tumor heterogeneity in the small cell lung cancer according to claim 1, wherein in the step 2, the HC subgroup and the ML subgroup are compared with the conventional TF typing and the NE typing respectively, and the HC subgroup and the ML subgroup are better than the conventional TF typing and the NE typing in distinguishing prognosis of patients.

5. The method for interpreting the inter-tumor and intra-tumor heterogeneity in the small cell lung cancer according to claim 1, wherein in the step 3, after analyzing the difference of the immune microenvironment characteristics between the HC subgroup and the ML subgroup by using the immune infiltration evaluation algorithm, a cellular infiltration change result is obtained: ROI samples in the ML subgroup is significantly higher than the HC subgroup in terms of an infiltration degree of all T cells.

6. The method for interpreting the inter-tumor and intra-tumor heterogeneity in the small cell lung cancer according to claim 1, wherein in the step 4, before selecting and constructing the tumor heterogeneity index model THIM, a feature selection is carried out on 129 DEGs by using an automatic encoder and is repeated for 500 times, then top 10 groups of genes are selected as candidate genes, and the top 10 groups of candidate genes are verified by immunofluorescence staining, and finally the model is selected and constructed according to the 10 groups of candidate genes after verification, wherein the 10 groups of candidate genes comprise 4 HC subgroup specificities and 6 ML subgroup specificities.

7. (canceled)

8. The method for interpreting the inter-tumor and intra-tumor heterogeneity in the small cell lung cancer according to claim 1, further comprising:
applying the tumor heterogeneity index model THIM to predict prognosis and immunotherapy response of small cell lung cancer (SCLC) patients, thereby adjusting clinical treatment strategy for the SCLC patients to treat the SCLC patients.
